(19) United States
(12) Patent Application Publication
(10) Pub. No.: US 2008/0306035 A1
(43) Pub. Date: Dec. 11, 2008

(54) Methods and Compositions for Treating Female Infertility Using Clomiphene

(75) Inventor: Joseph S. Podolski, The Woodlands, TX (US)

Correspondence Address: HOWREY LLP - DC, C/O IP DOCKETING DEPARTMENT, 2941 FAIRVIEW PARK DR, SUITE 200, FALLS CHURCH, VA, 22042-2924, US

(73) Assignee: Repros Therapeutics Inc., The Woodlands, TX (US)

(21) Appl. No.: 11/997,858

(22) PCT Filed: Aug. 2, 2006
(86) PCT No.: PCT/US06/30053

§ 371 Date: Jun. 11, 2008

Related U.S. Application Data
(60) Provisional application No. 60/706,094, filed on Aug. 5, 2005.

(51) Int. Cl. A61K 31/57; A61K 31/137

(52) U.S. Cl. 514/171; 514/651

(57) ABSTRACT

The present invention relates to the use of compositions comprising clomiphene for treating female infertility. The invention is also directed to a regimen of treatment for female infertility using trans-clomiphene, a mixture of cis- and trans-clomiphene, and cis-clomiphene.

### CROSS REFERENCE TO RELATED APPLICATIONS

The present application claims the benefit of U.S. Provisional Patent Application No. 60/706,094, filed Aug. 5, 2005 which is incorporated herein by reference.

### FIELD OF THE INVENTION

The present invention relates to female infertility. More specifically, the present invention relates to a treatment regimen for female infertility using clomiphene.

### BACKGROUND OF THE INVENTION

Clomiphene citrate is well known for treatment of female anovulation. It is currently approved as a mixture of both the cis- and trans-isomers, the cis-isomer being present as about 30% to 50% (Merck Manual) for fertility enhancement in the anovulatory patient. Clomiphene is believed to improve ovulation by initiating a series of endocrine events culminating in a preovulatory gonadotropin surge and subsequent follicular rupture. The anti-estrogenic properties of trans-clomiphene have been thought to precipitate the surge of luteinizing hormone (LH) associated with ovulation.

Standard treatment regimens of up to 100 mg of clomiphene citrate for 5 days per cycle have been associated with ovulation in about 70% of patients. Nevertheless, only about 25-40% of patients treated with clomiphene citrate achieve pregnancy. London, et al., Fertil. Steril. 73(3):620-626 (1999). Possible negative effects of clomiphene citrate treatment may include anti-estrogenic effects on the cervix and/or impairment of the development of the granulosa cells. The use of clomiphene citrate has also been linked to suboptimal endometrial thickness, which is associated with an elevated rate of preclinical abortion (loss of pregnancy with hCG ≧25 mIU prior to ultrasound observation of a sac). Dickey, et al., Hum. Reprod. 11(12):2623-2628 (1996).

The safety and effectiveness of clomiphene citrate has been widely evaluated with respect to use in the anovulatory patient. Although some studies have suggested that clomiphene citrate possesses both genotoxic and tumor enhancement effects, others have found no significant relationship between clomiphene treatment and cancer or fetal abnormalities. Clomiphene has been associated with side effects including: blurred vision, abodominal pain, bloating, blurred vision or other vision problems, hot flashes, breast discomfort, headache, dizziness or lightheadedness, heavy menstrual periods or bleeding between periods, mental depression, nausea, vomiting, nervousness, restlessness, fatigue, and insomnia. Nevertheless, clomiphene is commonly prescribed, alone or in combination with gonadotropins such as FSH or hCG, for treatment of unexplained infertility as well as a variety of identified fertility disorders.

Methods of treating the anovulatory patient with increased rates of pregnancy and delivery are desirable.

### SUMMARY OF THE INVENTION

Female infertility may be treated by administering compositions comprising clomiphene, which may comprise (cis, -Z-, trans-clomiphene) (hereinafter “cis-clomiphene”), (trans-, E-, cis-clomiphene) (hereinafter “trans-clomiphene”), and combinations thereof, or pharmaceutically acceptable salts thereof. The compositions may be administered at different times in the fertility cycle. The compositions may be administered in single or multiple daily doses, such as 1, 2, 3, 4, 5, 6, 7, 8, 9 or 10 daily doses.

A first composition comprising clomiphene may be administered prior to ovulation. The first composition may be administered starting between approximately the second and fifth day of the menstrual cycle, at the convenience of the amenorrheic patient, or at the recommendation of the treating physician. The clomiphene of the first composition may be trans-clomiphene, may be comprised essentially of trans-clomiphene, or may be comprised of more than about 70% trans-clomiphene. The clomiphene of the first composition may be administered in a dose from about 0.5 to about 100 mg. The dose of the first composition may also be from about 12.5 to about 50 mg clomiphene. The dose of the first composition may also be 12.5, 25 or 50 mg clomiphene. The first composition may be administered in a single or multiple doses. The first composition may be administered for approximately five days.

A second composition comprising clomiphene may be administered starting at ovulation and up to fertilization. The second composition may comprise a mixture of cis- and trans-clomiphene, in percentage of approximately 50-80% cis-clomiphene and 20-50% trans-clomiphene. The clomiphene of the second composition may be administered in a dose from about 0.5 to about 100 mg. The dose of the second composition may also be from about 0.5 to about 10 mg clomiphene. The dose of the second composition may also be 12.5, 25 or 50 mg clomiphene. The second composition may be administered in a single or multiple doses. If multiple dosages of the second composition are administered, at least one of the successive doses or all successive doses may have increasing percentage proportions of cis-clomiphene to trans-clomiphene.

A third composition comprising clomiphene may be administered after fertilization. The clomiphene of the third composition may be cis-clomiphene, may consist essentially of cis-clomiphene, or may be comprised of at least 50% cis-clomiphene. The clomiphene of the third composition may also be comprised of trans-clomiphene. The third composition may be administered in combination with progesterone. The clomiphene of the third composition may be administered in a dose from about 0.5 to about 100 mg. The dose of the third composition may also be from about 0.5 to about 10 mg clomiphene. The dose of the third composition may also be 12.5, 25 or 50 mg clomiphene. The third composition may be administered through part or all of the first trimester.

### BRIEF DESCRIPTION OF THE DRAWING

FIG. 1 shows the chemical structure of clomiphene citrate.

### DETAILED DESCRIPTION

The present invention is related to treating female infertility by administering clomiphene-containing compositions. As described above, clomiphene citrate (30-50% cis and 50-70% trans) is used in infertility treatment regimens. Although the anti-estrogenic activity of trans-clomiphene may be useful in stimulating ovulation, the estrogenic activity of cis-clomiphene may support and improve the maintenance of a fertilized embryo.

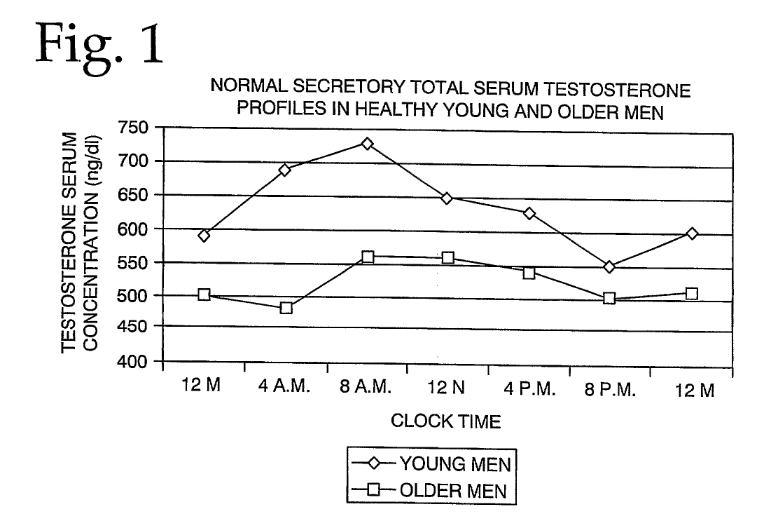

Trans-Clomiphene (FIG. 1) is an antiestrogen related to tamoxifen that is thought to operate in part by blocking the normal estrogen feedback on the hypothalamus and subsequent negative feedback on the pituitary. This results in an increased release of GnRH by the hypothalamus, which leads to increases in luteinizing hormone (LH) and follicle stimulating hormone (FSH). In women, these increased levels of gonadotropins are necessary for the recruitment, growth, and eventual rupture of a mature follicle (ovulation).

Ernst et al. have also noted that (the trans-isomer) is antiestrogenic (AE) while the cis-isomer is the more potent and more estrogenic form and has also been reported to have anti-estrogenic activity. The authors attribute the effect of clomiphene citrate on ovulatory activity to both forms stating that the mixture is more effective than trans-clomiphene alone. The trans-isomer aids ovulation at the level of the hypothalamus. The estrogenic isomer cis-clomiphene contributes to enchanced ovulation elsewhere in the physiologic pathway leading to ovulation. The isomers are also reported to have different in vivo half-life. Furthermore the cis form has been reported to leave residual blood levels for in excess of one month following a single dose.

As described above, clomiphene has multiple types of activity, including but not limited to estrogenic and/or antiestrogenic effects on the pituitary, the hypothalamus, and the ovary itself. Therefore, clomiphene may have a variety of direct and indirect effects in a patient. For example, the administration of clomiphene may prevent or cure luteal defects or insufficiencies, which may be due to abnormal progesterone levels. The administration of clomiphene may also augment uterine blood flow, which may assist in development of the endometrium. Both cis-clomiphene and trans-clomiphene have been shown to raise uterine blood flow. Although both cis- and trans-clomiphene take longer to achieve peak blood flow than estradiol, the duration of the effect is greater than for estradiol. Trans-clomiphene may raise uterine blood flow to higher peak levels than estradiol or cis-clomiphene, but it must be administered at much higher doses to have this effect. The development of the endometrium may increase the likelihood of successful implantation of a fertilized egg. Additionally, the estrogenic activity of cis-clomiphene may prevent or improve thickening of the cervical mucus caused by the administration of trans-clomiphene. By counteracting the thickening of the cervical mucus often associated with the anti-estrogenic effects of clomiphene, cis-clomiphene may improve the hospitability of the cervix to sperm and improve the chance of fertilization.

### 1. Compositions and Activity

A first composition comprising clomiphene may be administered prior to ovulation. The clomiphene of the first composition may be comprised of at least about 70% of trans-clomiphene. Prior to ovulation, the use of trans-clomiphene may be an effective means of increasing LH while potentially decreasing the side effects suffered by the patient during traditional clomiphene citrate treatment. The first composition may be administered in one or more doses.

A second composition comprising clomiphene may be administered starting at ovulation and up to fertilization. The clomiphene of the second composition may be comprised of cis- and trans-clomiphene. The second composition may be administered in one or more doses. If multiple doses of the second composition are administered, at least one or each successive dose may be comprised of increasing proportions of cis-clomiphene relative to trans-clomiphene. The second composition may provide improved hospitability of the cervix to sperm, which may lead to improved fertilization, and increased uterine blood flow, which may improve the susceptibility of the endometrium to implantation by the fertilized ovum. The skilled clinician may consider, for example, the patient's estradiol levels and endometrial thickness to determine the proper dosage. The second composition may be useful in patients having lower than normal estradiol levels and/or insufficient endometrial thickness. The second composition may also treat a luteal insufficiency.

A third composition comprising clomiphene may be administered beginning at fertilization, which may be by intercourse or artificial insemination, or upon the transfer of embryos into the patient's uterus. The clomiphene of the third composition may be comprised of at least 50% cis-clomiphene.

The third composition may provide increased uterine blood flow, which may improve the susceptibility of the endometrium to implantation by the fertilized ovum. The skilled clinician may consider, for example, the patient's estradiol levels and endometrial thickness to determine the proper dosage, but it is contemplated that the third composition will be especially useful in patients having lower than normal estradiol levels and/or insufficient endometrial thickness.

The third composition may comprise trans-clomiphene, which may be beneficial, such as in cases where the patient's progesterone levels are insufficient, or where a prior luteal insufficiency is not fully resolved.

### 2. Dosing and Administration Regimen

The first composition may be administered as part of an ovulation induction regimen. The clomiphene of the first composition may be consisting of trans-clomiphene, may be comprised essentially of trans-clomiphene, or may be comprised of at least about 70% trans-clomiphene. The dosage in which the clomiphene of the first composition is administered may be about 25-200 mg/day, for about five days, starting at or around days 2-5 of the menstrual cycle, at the convenience of the amenorrheic or oligomenorrheic patient, or at the recommendation of the treating physician.

The second composition may be administered starting at or after ovulation as detected by basal body temperature, LH surge, or by other means. Dosages of clomiphene may range from about 10 μg/kg to 10 mg/kg to more than 10 mg/kg. The second composition may comprise both cis- and trans-clomiphene and may comprise more than about 50% cis-clomiphene. The second composition may comprise multiple formulations throughout the treatment regimen, with at least one to each subsequent formulation having an increased proportion of cis-clomiphene. For example, where the second composition is administered in two formulations, one formulation may comprise less than about 50% cis-clomiphene and the other formulation may comprise more than about 50% cis-clomiphene. In another example, where the second composition is administered in three formulations, one formulation may comprise about 50% cis-clomiphene, while a second formulation may comprise about 75% cis-clomiphene, and a third formulation may comprise about 75% cis-clomiphene. Other formulations and dosage regimens are also contemplated and will be understood by one skilled in the art.

The third composition may be administered starting at or after insemination or fertilization as detected by hCG ≧25 mIU, or by ultrasound. The third composition may comprise cis-clomiphene or consist essentially of cis-clomiphene. In some patients, the third composition may further comprise one or more additional components. For example, some patients whose corpus luteum is insufficient (i.e., does not produce adequate progesterone) it may be beneficial to augment progesterone through some or all of the first trimester until the placenta is able to produce adequate progesterone to support the pregnancy. In such cases, cis-clomiphene could be administered alone, in combination with small amounts of trans-clomiphene, or, alternatively, in combination with progesterone. Dosages may range from about 10 μg/kg to 10 mg/kg to more than 10 mg/kg clomiphene. The duration of treatment may be determined based on the patient's hormone levels, medical history, and/or the discretion of the treating physician.

### 3. Formulations

Suitable pharmaceutical compositions or unit dosage form may be in the form of solids, such as tablets or filled capsules or liquids such as solutions suspensions, emulsions, elixirs or capsules filled with the same, all for oral use. The compositions may also be in the form of sterile injectable solutions or emulsions for parenteral (including subcutaneous) use. Such pharmaceutical compositions and unit dosage forms thereof may comprise ingredients in conventional proportions.

Compositions according to the present invention may also be administered by the intravenous, subcutaneous, buccal, transmucosal, intrathecal, intradermal, intracisternal or other routes of administration. During the course of treatment, hormone levels may be measured as described above and dosages may be altered to achieve a sufficient change in FSH, LH, estrogen, progesterone or other hormones to achieve the desired physiological results associated with pregnancy described above. The compositions may be administered daily, non-daily or episodic. For example, the compositions may be administered at a dosing regimen of 1, 2, 3, 4, 5, 6, 7, 8, 9, 10, 11, 12, 13 or 14 days between administrations.

The following Example is meant to be illustrative of the invention and is not intended to limit the scope of the invention as set out is the appended claims.

### EXAMPLE 1

### Use of Clomiphene Compositions in an Infertility Treatment Regimen

A first composition comprising purified trans-clomiphene (50 mg) is administered daily to an anovulatory adult female for 5 days starting on the third day of the menstrual cycle. The second composition is administered in two daily 0.5 mg doses starting at ovulation as detected by basal body temperature. The first dosage of the second composition comprises approximately 51% cis-clomiphene and 49% trans-clomiphene. The second dosage of the second composition comprises approximately 75% cis-clomiphene and 25% trans-clomiphene. When the patient's hCG levels reach levels associated with pregnancy (hCG ≧25 mIU), the third composition is administered in a single dosage.

Upon completion treatment with the first, second, and third clomiphene compositions, the adult female successfully achieves and sustains a pregnancy.

1. A method of treating female infertility comprising:
(a) administering to a patient in need thereof prior to ovulation a first composition comprising clomiphene, wherein the clomiphene is comprised of at least about 70% trans-clomiphene;
(b) administering to the patient between ovulation and fertilization a second composition comprising clomiphene, wherein the clomiphene is comprised of trans-clomiphene and cis-clomiphene; and optionally
(c) administering to the patient after fertilization a third composition comprising clomiphene, wherein the clomiphene is comprised of at least about 50% cis-clomiphene.

2. The method of claim 1, wherein the compositions are administered in a single or multiple daily doses.

3. The method of claim 2, wherein the number of daily doses is selected from the group consisting of 1, 2, 3, 4, 5, 6, 7, 8, 9 and 10.

4. The method of claim 1, wherein the first composition is administered starting at about day 2 to about day 5 of the patient's menstrual cycle.

5. The method of claim 1, wherein the clomiphene of the first composition consists essentially of trans-clomiphene.

6. The method of claim 1, wherein the clomiphene of the first composition is trans-clomiphene.

7. The method of claim 2, wherein the first composition is administered in multiple doses.

8. The method of claim 2, wherein the first composition is administered in a single dose.

9. The method of claim 1, wherein the clomiphene of the second composition is comprised of about 50-80% of cis-clomiphene and about 20-50% of trans-clomiphene.

10. The method of claim 2, wherein the second composition is administered in multiple doses.

11. The method of claim 10, wherein at least one successive dose comprises an increased percentage of cis-clomiphene compared to the previous dose.

12. The method of claim 2, wherein the second composition is administered in a single dose.

13. The method of claim 1, wherein the clomiphene of the third composition consists essentially of cis-clomiphene.

14. The method of claim 1, wherein the clomiphene of the third composition is cis-clomiphene.

15. The method of claim 1, wherein the clomiphene of the third composition is comprised of trans-clomiphene.

16. The method of claim 1, wherein the third composition further comprises progesterone.

17. The method of claim 2, wherein the third composition is administered in a single dose.

18. The method of claim 2, wherein the third composition is administered in multiple doses.

19. The method of claim 1, wherein the third composition is administered throughout the first trimester of the patient's pregnancy.
